(19) United States
(12) Patent Application Publication
(10) Pub. No.: US 2023/0170045 A1
(43) Pub. Date: Jun. 1, 2023

(54) COMPREHENSIVE POLYGENIC RISK PREDICTION FOR BREAST CANCER

(71) Applicant: Myriad Genetics, Inc., Salt Lake City, UT (US)

(72) Inventors: Shannon Gallagher, Salt Lake City, UT (US); Elisha Hughes, Salt Lake City, UT (US); Alexander Gutin, Salt Lake City, UT (US); Jerry Lanchbury, Salt Lake City, UT (US)

(73) Assignee: Myriad Genetics, Inc., Salt Lake City, UT (US)

(21) Appl. No.: 17/920,012

(22) PCT Filed: Apr. 16, 2021
(86) PCT No.: PCT/US2021/027651

§ 371 Date: Oct. 19, 2022

Related U.S. Application Data
(60) Provisional application No. 63/012,704, filed on Apr. 20, 2020.

(51) Int. Cl. G16B 20/20; C12Q 1/6886; G16H 50/30

(52) U.S. Cl. CPC G16B 20/20; C12Q 1/6886; G16H 50/30; C12Q 2600/156

(57) ABSTRACT

Provided herein are methods for determining a polygenic risk score and breast cancer estimated risk for medical use, as well as for treating breast cancer. Methods of this invention can provide a polygenic risk score which takes into account a plurality of breast cancer associated SNP markers. Also provide is a comprehensive breast cancer risk estimation with increased accuracy.

### TECHNICAL FIELD

This invention relates to the fields of genetics and medicine. More particularly, this invention relates to methods for assessing and predicting polygenic traits and breast cancer risks for medical use, as well as treating breast cancer.

### BACKGROUND

It is desirable to use polygenomic risk scores to assess the expectation of a clinical trait or condition such as cancer. Risk scores from genomic data depend on identifying polymorphic loci to be used.

Conventional methods for breast cancer have identified various breast cancer associated genes. However, germline pathogenic variants of breast cancer associated genes introduce complexity that diminishes the accuracy and predictive power of conventional methods. A drawback of conventional methods is lack of a comprehensive analysis which combines breast cancer associated genes with polygenomic-type risk scores and other risk factors.

Breast cancer risk for carriers of breast cancer associated genes must be assessed to stratify patient risk levels. It is desirable to provide risk assessment over various time periods such as 10-year and lifetime risk prediction. Risk stratification can be used by caregivers to inform individualized patient decision-making, as well as for targeting the screening, prevention and treatment of breast cancer. Drawbacks of conventional methods include limited accuracy of risk stratification and prediction which confuses prevention and treatment strategies and jeopardizes patient outcomes.

Further drawbacks of conventional methods, even when polygenomic risk scores based on SNP analysis is combined with moderate to highly penetrant breast cancer genes, include large inaccuracies because effect sizes may be different for different marker genes.

What is needed is a highly accurate and comprehensive method for determining polygenic risk scores for breast cancer with reduced errors in predictive ability. An advantageous clinical risk algorithm can improve medical care and patient treatment.

There is an urgent need for methods to assess risk of breast cancer. There is a need for methods that can be efficiently brought to the point of medical care.

### BRIEF SUMMARY

This invention provides methods for determining polygenic traits and risks for breast cancer. The methods of this invention can be used in medicine, as well as for treating diseases for which risk is identified and/or assessed.

In some aspects, methods of this invention may provide superior prediction of clinical risk in breast cancer patients. The methods of this invention can provide polygenic risk prediction for breast cancer which comprehensively takes into account a wide range of risk factors.

A comprehensive approach of this invention may take into account three or more classes of risk markers and elements.

A comprehensive approach can include any number of the more than 10,000 individual pathogenic variants (PV) in genes BRCA1 and BRCA2.

A comprehensive approach can further include any number of individual pathogenic variants in breast cancer susceptibility genes such as PALB2, CHEK2, and ATM, which are about as prevalent as for BRCA1 and BRCA2.

A comprehensive approach may include risk marker variants, which may be single nucleotide polymorphisms (SNP). SNPs and other variants have been associated with breast cancer risk in large whole-genome association studies. Combinations of SNPs can be aggregated into a polygenic risk score (PRS) which can stratify unaffected women for breast cancer risk, irrespective of the presence or absence of a family history of the disease.

Additional classes of markers or elements can include age, family history, breast density, and hormone exposure.

In certain aspects, the clinical utility of this invention includes superior prediction of clinical risk for breast cancer patients having European ancestry.

In some aspects, methods of this invention can provide a polygenic score which accounts for penetrant genes associated with breast cancer.

A polygenic score obtained by the methods of this invention can provide surprisingly increased accuracy in determining breast cancer risks.

Methods of this invention can provide surprisingly accurate determination of polygenic traits and risks by comprehensively assessing and including contributions of a wide range of markers for breast cancer.

Embodiments of this invention contemplate determining the levels of polygenic traits and risks in the form of a score based on various genomic risk loci. The genomic risk loci can be discretely identified and defined, so that accurate determination can be done by genotyping subjects.

In certain aspects, the genomic risk loci can include genomic risk markers for breast cancer, which are combined with additional risk markers that can be specifically breast cancer-informative.

Embodiments of this invention include:

A method for assessing breast cancer risk in a subject having a pathogenic variant in a breast cancer associated gene, the method comprising:

measuring a genotype of the subject; and

calculating a polygenic risk score for breast cancer risk for the subject based on a plurality of breast cancer associated SNP markers of the genotype and additional variables for age, personal cancer history, family cancer history, and ancestry of the subject.

The method above, further comprising

calculating an adjusted TC risk for the subject; and

assessing comprehensive breast cancer risk in the subject by combining the combined polygenic risk score and adjusted TC risk, which may optionally be done with a clinical cohort.

The method above, further comprising validating the breast cancer risk in a clinical or comparative cohort.

The method above, wherein the genotype is measured by NGS.

The method above, wherein the genotype is determined with a sequencing chip.

The method above, wherein the plurality of breast cancer associated SNP markers is from 10 to 10,000 SNP markers.

The method above, wherein the plurality of breast cancer associated SNP markers is from 50 to 200 SNP markers.

The method above, wherein the adjusted TC risk, TC*, is calculated to account for the presence of a CHEK2 DM according to Equation I:

TC*=1−(1−TC)^{exp(β}^{_{CHEK2}}^{+k}^{ᵢ}⁾, for family history strata i Equation I;

wherein TC is the standard lifetime risk as calculated by Tyrer-Cuzick version 7.02; β_{CHEK2} is a log-odds ratio for CHEK2 carriers as a predictor of breast cancer risk; kᵢ is a calibration constant for a specific family history strata i;
wherein subjects are divided into strata based on relative risk based on a comparison of individual risk due to familial cancer history compared to general population risk; wherein constants kᵢ can be calculated so that the mean of exp(β_{CHEK2}× [symbol] _{CHEK2}) within each strata is 1.

The method above, wherein the adjusted TC risk includes factors for age, body mass index, age at menarche, obstetric history, age at menopause, history of a benign breast condition that increases breast cancer risk such as hyperplasia, atypical hyperplasia, and/or LCIS, history of ovarian cancer, use of hormone replacement therapy, family history of breast and ovarian cancer, and Ashkenazi inheritance.

The method above, wherein the comprehensive breast cancer risk is a relative risk score (ComprehensiveRRS) for breast cancer risk made using an adjusted Tyrer-Cuzick risk and taking into account the presence of a CHEK2-DM according to Equation II;

ComprehensiveRRS=1−(1−TC*)^{exp(β}^{_{RRS}}^{+c}^{ᵢ}⁾ for family history strata i Equation II;
wherein TC* is the adjusted Tyrer-Cuzick risk after accounting for the CHEK2 DM, β_{RRS} is the log-odds per-unit log odds ratio of a polygenic SNP score from a multivariable logistic regression model with the effect of breast cancer family history fixed, and cᵢ is a calibration constant for a specific family history strata i, calculated such that the average relative risk due to the polygenic SNP score was 1 within unaffected subjects from strata kᵢ.

The method above, wherein the genotype identifies a subject having the presence of a CHEK2-DM.

The method above, wherein the genotype identifies a subject who tested negative for mutations in breast cancer associated genes comprising BRCA1, BRCA2, TP53, PTEN, STK11, CDH1, PALB2, ATM, NBN, and BARD1.

The method above, wherein the calculating a polygenic risk score comprises a linear combination of centered risk alleles according to Equation III.

Polygenic Risk Score=b₁(x₁−u₁)+b₂(x₂−u₂)+ . . . . +b_{N}(x_{N}−u_{N}) Equation III;

where N is the total number of SNPs selected;
the coefficient bₖ is the per-allele log OR for breast cancer association of the kth SNP estimated from meta-analysis of literature and the development cohort;
xₖ is the number of alleles of the kth SNP carried by an individual patient which is 0, 1 or 2; and uₖ is the average number of alleles of the kth SNP reported for individuals included in large general population studies.

The method above, wherein the total number of SNPs is 86, which may be the SNPs in Table 1.

The method above, wherein the clinical data used for the calculation optionally includes women of white/non-hispanic and/or Ashkenazi Jewish ancestry.

A method for recommending therapy for a subject having a pathogenic variant in a breast cancer associated gene and having breast cancer or at risk of breast cancer, the method comprising:

measuring a genotype of the subject;

calculating a polygenic risk score for breast cancer risk for the subject based on a plurality of breast cancer associated SNP markers of the genotype and additional variables for age, personal cancer history, family cancer history, and ancestry of the subject; and

recommending a therapy for the disease based on the risk score indicating a need for a therapy or exceeding a threshold level.

The method above, further comprising

calculating an adjusted TC risk for the subject; and

assessing comprehensive breast cancer risk in the subject by combining, optionally by single regression, the polygenic risk score and the adjusted TC risk, optionally using a clinical cohort.

The method above, further comprising validating the breast cancer risk in a clinical or comparative cohort.

The method above, wherein the therapy is one of:

a therapy for the disease;

a monitoring period followed by a therapy for the disease;

a tapering of a therapy for the disease.

The method above, wherein the therapy is one or more of surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound.

A method for identifying a subject having breast cancer who benefits from a treatment, the method comprising:

measuring a genotype of the subject;

calculating a polygenic risk score for breast cancer risk for the subject based on a plurality of breast cancer associated SNP markers of the genotype and additional variables for age, personal cancer history, family cancer history, and ancestry of the subject; and

identifying the subject having the disease who benefits from a treatment for the disease based on the risk score, which may indicate a need for a therapy, or may exceed a threshold level.

The method above, further comprising

calculating an adjusted TC risk for the subject; and

assessing comprehensive breast cancer risk in the subject by combining the polygenic risk score and the adjusted TC risk, optionally using a clinical cohort.

The method above, further comprising validating the breast cancer risk in a clinical or comparative cohort.

The method above, wherein the therapy is one of:

a therapy for the disease;

a monitoring period followed by a therapy for the disease;

a tapering of a therapy for the disease.

The method above, wherein the therapy is one or more of surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound.

A method for treating a disease in a subject in need thereof, the method comprising:

measuring a genotype of the subject;

calculating a polygenic risk score for breast cancer risk for the subject based on a plurality of breast cancer associated SNP markers of the genotype and additional variables for age, personal cancer history, family cancer history, and ancestry of the subject;

identifying the subject having the disease who benefits from a treatment for the disease based on the risk score, which may indicate a need for a therapy, or exceed a threshold level; and administering to the subject one of:

a therapy for the disease;

a monitoring period followed by a therapy for the disease;

a tapering of a therapy for the disease.

The method above, wherein the therapy is a cancer therapy selected from one or more of surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound.

A method for monitoring a response of a subject having a disease, the method comprising:

measuring a genotype of the subject;

calculating a polygenic risk score for breast cancer risk for the subject based on a plurality of breast cancer associated SNP markers of the genotype and additional variables for age, personal cancer history, family cancer history, and ancestry of the subject.

The method above, further comprising

calculating an adjusted TC risk for the subject; and

assessing comprehensive breast cancer risk in the subject by combining the polygenic risk score and the adjusted TC risk with a clinical cohort.

The method above, further comprising validating the breast cancer risk in a clinical or comparative cohort.

A method for prognosing a subject having a disease, the method comprising:

measuring a genotype of the subject;

calculating a polygenic risk score for breast cancer risk for the subject based on a plurality of breast cancer associated SNP markers of the genotype and additional variables for age, personal cancer history, family cancer history, and ancestry of the subject; and

prognosing the subject as having a poor prognosis for the disease based on the risk score, which may indicate a need for therapy, or may exceed a threshold level.

The method above, further comprising

calculating an adjusted TC risk for the subject; and

assessing comprehensive breast cancer risk in the subject by combining, optionally by single regression, the polygenic risk score and the adjusted TC risk, optionally using a clinical cohort.

The method above, further comprising validating the breast cancer risk in a comparative cohort.

A system for assessing risk of a disease in a subject, the system comprising:

a processor for receiving a genotype of the subject;

one or more processors for carrying out the steps:
calculating a polygenic risk score for breast cancer risk for the subject based on a plurality of breast cancer associated SNP markers of the genotype and additional variables for age, personal cancer history, family cancer history, and ancestry of the subject; and
calculating an adjusted TC risk for the subject;

assessing comprehensive breast cancer risk in the subject by combining the polygenic risk score and adjusted TC risk; and

a display for displaying and/or reporting the risk score.

A non-transitory machine-readable storage medium having stored therein instructions for execution by a processor which cause the processor to perform the steps of a method for assessing risk of a disease in a subject, the method comprising:

receiving a genotype of the subject;

calculating a polygenic risk score for breast cancer risk for the subject based on a plurality of breast cancer associated SNP markers of the genotype and additional variables for age, personal cancer history, family cancer history, and ancestry of the subject;

calculating an adjusted TC risk for the subject;

assessing comprehensive breast cancer risk in the subject by combining the polygenic risk score and adjusted TC risk; and

sending to a processor output for displaying and/or reporting the risk score.

### BRIEF DESCRIPTION OF THE DRAWINGS

FIG. 1 shows a comparison of a Comprehensive-RRS method for breast cancer risk as compared to a polygenic method based on 86-SNP markers. The change in the likelihood ratio test values (LRT) for breast cancer prediction was shown. The remaining lifetime breast cancer risk was determined for study individuals having CHEK2 mutations. The change in LRT shows that risk scores were significantly modified by a polygenic scoring method based on 86-SNP markers. To compare the 86-SNP marker method and the Comprehensive-RRS method, one multivariate logistic regression was performed for predicting breast cancer status, with age at testing and Ashkenazi ancestry as covariates. Effect sizes were calculated as odds ratios per one-unit standard deviation. As shown in FIG. 1, the scores showed greater discrimination for breast cancer diagnosis for the Comprehensive-RRS method as compared to the polygenic method based on 86-SNP markers.

FIG. 2 shows lifetime breast cancer risk as probability density function against absolute risk estimates by age 80 for carriers of pathogenic variants (PV) in breast cancer associated genes as modified by an 86-SNP score method. For women with pathogenic variants (PV) in moderate-risk breast cancer genes CHEK2, ATM, and PALB2, point estimates were higher than for BRCA1/2 carriers. The interaction between the 86-SNP score and gene carrier type was significant. The most pronounced risk discrimination was observed for CHEK2 carriers, where the effect size was equivalent to the odds ratios observed in non-carriers and for the general population.

FIG. 3 shows standardized odds ratios for association between an 86-SNP score method and personal breast cancer history for carriers of each gene and non-carriers. FIG. 3 shows a forest plot of the standardized odds ratio for the association between the 86-SNP score and personal breast cancer history along with 95% confidence intervals for carriers of each gene and non-carriers.

FIG. 4 shows observed (solid lines) versus expected (dashed lines) odds ratios per percentile of an 86-SNP score method by carrier gene.

FIG. 5 shows odds ratios for the association of an 86-SNP score method with the risk of developing breast cancer by age bin and carrier gene.

FIG. 6 shows odds ratios for the association of an 86-SNP score method with breast cancer risk by family history (X markers represent without breast cancer) and carrier gene (filled square markers represent with breast cancer).

FIG. 7 shows odds ratios for the association of an 86-SNP score method with breast cancer risk by weighted relative count and carrier gene.

### DETAILED DESCRIPTION OF THE DISCLOSURE

This invention includes methods for polygenic risk prediction to provide comprehensive risk assessment for breast cancer.

In some aspects, this invention provides methods for polygenic risk prediction with increased accuracy of risk assessment for breast cancer.

Embodiments of this invention further provide reliable breast cancer risk associations based on populations of European women.

This disclosure provides various methods for clinical risk management, risk magnitude assessment, as well as polygenic risk scores, and non-clinical trait prediction. Methods of this invention can provide predictive ability that is surprisingly accurate for primarily European genotypes.

Aspects of this disclosure include genotyping variant loci and combining the genotypes in the form of a polygenic score to predict risk of a clinical condition or an extent of manifestation of a biological trait.

In further embodiments, a plurality of trait risk markers can be used along to provide a polygenic risk prediction for the trait.

In further embodiments, the plurality of trait risk markers may include from 1-100 low to moderately penetrant breast cancer gene markers, or from 1-20 low to moderately penetrant breast cancer gene markers, or from 1-10 low to moderately penetrant breast cancer gene markers.

In certain embodiments, the plurality of trait risk markers may include from 1-10,000 SNP markers, or from 1-1000 SNP markers, or from 1-100 SNP markers. A plurality of trait risk markers may be from 1-1000 breast cancer SNP markers, or from cancer 1-500 breast cancer SNP markers, or from 1-100 breast cancer SNP markers.

In additional embodiments, the plurality of trait risk markers may include from 1-100 family history elements, or from 1-20 family history elements, or from 1-10 family history elements.

Embodiments of this invention may include a plurality of trait risk markers such as from 1-100 clinical elements, or from 1-20 clinical elements, or from 1-10 clinical elements.

Embodiments herein can provide improved polygenic risk prediction for breast cancer.

Comprehensive risk assessment combining a polygenic SNP scoring method with other risk factors and elements can improve the accuracy of risk estimates and facilitate decision-making for women with pathogenic variants in moderately penetrant genes.

Embodiments of this invention provide comprehensive risk assessment that can overcome drawbacks of conventional methods, such as inaccuracies of differing effect sizes for different marker genes.

Further aspects of this invention can provide unique methods for determining a breast cancer risk score.

In further aspects, a polygenic risk score of this invention may be surprisingly more accurate for breast cancer than using conventional methods.

Aspects of this invention can provide a comprehensive risk prediction for women of European ancestry. Comprehensive risk prediction can provide the level and/or stratification of remaining lifetime risk in a subject, or 10 year risk.

In certain methods of this invention, surprisingly precise and validated risk score estimates for breast cancer in women who carry PVs in both high and moderate risk genes can be provided. Such comprehensive risk estimates can allow individually tailored medical care.

The methods of this invention can surprisingly improve the accuracy and/or precision of risk estimates in subjects having pathogenic variants of low to moderately penetrant breast cancer genes.

Some aspects of this invention include methods for assessing a validated, comprehensive risk of breast cancer using a polygenic SNP score in combination with breast cancer associated genes such as BRCA1, BRCA2, ATM, CHEK2 and PALB2, among others, along with other markers and elements as described above.

Further aspects of this invention include methods for assessing a validated, comprehensive risk of breast cancer using a polygenic SNP score in combination with breast cancer associated gene CHEK2-DM having a deleterious mutation, wherein the study excluded subject having pathogenic mutation in other breast cancer associated genes including BRCA1, BRCA2, TP53, PTEN, STK11, CDH1, PALB2, ATM, NBN, and BARD1, along with other markers and elements as described above.

In certain aspects, an association between the polygenic risk scores and breast cancer may be evaluated by fixed stratification methods. The fixed stratification may be adjusted for age and family history, among other variables and elements.

Embodiments of this invention can provide women having pathogenic variants in low to moderately penetrant genes an estimated lifetime risk for breast cancer with increased accuracy. Such risk estimation is useful to inform decisions based on a threshold for more aggressive screening, including consideration of breast magnetic resonance imaging (MRI).

In some aspects, disclosed herein are methods that can utilize low to moderately penetrant cancer genes along with breast cancer SNP markers to provide a comprehensive polygenic risk score for breast cancer.

In additional aspects, this invention provides methods that can utilize low to moderately penetrant cancer genes, breast cancer SNP markers, and Tyrer-Cuzick variables to provide a comprehensive risk estimation score for breast cancer.

In further aspects, this invention provides methods that can utilize low to moderately penetrant cancer genes, breast cancer SNP markers, Tyrer-Cuzick variables, and additional family history (FH) variables to provide a comprehensive polygenic risk estimation score for breast cancer.

In further aspects, this invention provides methods that can utilize CHEK2, breast cancer SNP markers, and other Tyrer-Cuzick variables, along with additional family history variables to provide a surprisingly accurate polygenic risk estimation score for remaining lifetime risk of breast cancer.

Some examples of breast cancer risk markers are given in: Prediction of breast cancer risk based on profiling with common genetic variants, Mavaddat et al., J Natl Cancer Inst., 2015, April 8, Vol. 107(5), djv036.

Some examples of breast cancer risk markers are given in: Michailidou et al., Genome-wide association analysis of more than 120,000 individuals identifies 15 new susceptibility loci for breast cancer, Nat Genet., 2015, Vol. 47, pp. 373.

Some examples of breast cancer risk markers are given in Characterizing Genetic Susceptibility to Breast Cancer in Women of African Ancestry, Feng et al., Cancer Epidemiol Biomarkers Prev., 2017, July, Vol. 26(7), pp. 1016-1026.

Some examples of breast cancer risk markers are given in Rainville, I. et al., Breast Cancer Research and Treatment, 2020, Vol. 180, pp. 503-509.

Some examples of breast cancer risk markers are given in Early Diagnosis of Breast Cancer, Wang et al., Sensors (Basel), 2017, July, Vol. 17(7), p. 1572.

Some examples of genetic modifiers for breast cancer risk are given in Muranen T A, et al., Genetics in Medicine, 2017, Vol. 19(5), pp. 599-603.

Some examples of risk scores for breast cancer are given in Kuchenbaecker K, et al., J Natl Cancer Inst., 2017, Vol. 109(7), djw302.

Some examples for cancer risk are given in: Perencevich M, et al., Gastroenterology & Hepatology, 2011, Vol. 7(6), pp. 420-423.

Some examples for gene analysis are given in: Lek et al., Nature, 2016, Vol. 536.7616, pp. 285.

### Comprehensive Breast Cancer Risk Estimation

A comprehensive estimation of breast cancer risk can made for women who had a deleterious mutation (DM) in the CHEK2 gene, and who tested negative for mutations in all of 10 other breast cancer associated genes including BRCA1, BRCA2, TP53, PTEN, STK11, CDH1, PALB2, ATM, NBN, and BARD1.

In some embodiments, a comprehensive estimation of breast cancer risk can utilize an adjusted Tyrer-Cuzick remaining lifetime risk calculation, for example, adjusting Tyrer-Cuzick version 7.02. The risk estimation can include familial cancer history and personal risk factors obtained from a subject questionnaire. Subjects may be excluded if they had a personal history of LCIS, atypical hyperplasia, or breast biopsy.

Methods of this invention include assessing breast cancer risk in a subject having a pathogenic variant in a breast cancer associated gene by measuring a genotype of the subject, calculating a polygenic risk score for breast cancer risk for the subject based on a plurality of breast cancer associated SNP markers of the genotype and additional variables for age, personal cancer history, family cancer history, and ancestry of the subject, calculating an adjusted TC risk (TC*) for the subject, and combining the polygenic risk score and the adjusted TC risk to assess comprehensive breast cancer risk in the subject. The polygenic risk score for this combination can be determined with any number of SNPs, for example 20 or more, or 30 or more, or 50 or more SNPs. Examples of pertinent SNPs for a polygenic risk score include those in Table 1.

The Tyrer-Cuzick method can be used to estimate the likelihood of a woman developing breast cancer in 10 years, and over the course of her lifetime. An un-adjusted Tyrer-Cuzick method is given in Tyrer J, Duffy S W, Cuzick J., A breast cancer prediction model incorporating familial and personal risk factors, Stat. Med., 2004, Vol. 23(7), pp. 1111-1130.

The Tyrer-Cuzick method may take into account risk factors including age, body mass index, age at menarche, obstetric history, age at menopause, history of a benign breast condition that increases breast cancer risk such as hyperplasia, atypical hyperplasia, and/or LCIS, history of ovarian cancer, use of hormone replacement therapy, as well as family history including breast and ovarian cancer, Ashkenazi inheritance, and genetic testing if any.

An adjusted Tyrer-Cuzick risk (TC*) may be calculated to account for the presence of a CHEK2 DM using Equation I.

TC*=1−(1−TC)^{exp(β}^{_{CHEK2}}^{+k}^{ᵢ}⁾, for family history strata i Equation I.

where TC is the standard lifetime risk as calculated by Tyrer-Cuzick version 7.02, CHEK2 is a log-odds ratio for CHEK2 carriers as a predictor of breast cancer risk, and kᵢ is a calibration constant for a specific family history strata i. For this adjusted Tyrer-Cuzick risk, subjects can be divided into strata based on relative risk. Relative risk can be a comparison of individual risk due to familial cancer history compared to general population risk. The constants kᵢ can be calculated so that the mean of exp(β_{CHEK2}× [symbol] _{CHEK2}) within each strata is 1.

β_{CHEK2} can be determined in a cohort of subjects including individuals with CHEK2-DMs and subjects that are negative for gene mutations in a number of other breast cancer associated genes.

A comprehensive estimation of breast cancer risk can made using an adjusted Tyrer-Cuzick risk and taking into account the presence of a CHEK2-DM. A comprehensive estimation can be made for women who had a deleterious mutation (DM) in the CHEK2 gene, and who tested negative for mutations in all of 10 other breast cancer associated genes including BRCA1, BRCA2, TP53, PTEN, STK11, CDH1, PALB2, ATM, NBN, and BARD1.

A comprehensive relative risk score (ComprehensiveRRS) for breast cancer risk can made using an adjusted Tyrer-Cuzick risk and taking into account the presence of a CHEK2-DM according to Equation II.

ComprehensiveRRS=1−(1−TC*)^{exp(β}^{_{RRS}}^{+c}^{ᵢ}⁾ for family history stratai Equation II;

where TC* is the adjusted Tyrer-Cuzick risk after accounting for the CHEK2 DM, β_{RRS} is the log-odds per-unit log odds ratio of a polygenic SNP score from a multivariable logistic regression model with the effect of breast cancer family history fixed, and cᵢ is a calibration constant for a specific family history strata i, calculated such that the average relative risk due to the polygenic SNP score was 1 within unaffected women from strata kᵢ.

In some embodiments, the polygenic SNP score can be an 86-SNP polygenic risk score.

### Polygenic Cancer Risk Estimation

A polygenic estimation of breast cancer risk can made using an 86-SNP Polygenic Risk Score. A SNP Polygenic Risk Score can provide association with risk of breast cancer development in women carrying pathogenic variants in low to moderately penetrant genes such as ATM, CHEK2, and PALB2. The absolute risks of breast cancer to age 80 can be calculated to illustrate the potential clinical utility of polygenic stratification in women with pathogenic variants in BRCA1/2, ATM, CHEK2, and PALB2.

A polygenic risk score can be defined as a linear combination of centered risk alleles according to Equation III.

Polygenic Risk Score=b₁(x₁−u₁)+b₂(x₂−u₂)+ . . . +b_{N}(x_{N}−u_{N}) Equation III;

where N is the total number of SNPs selected, the coefficient bₖ is the per-allele log OR for breast cancer association of the kth SNP estimated from meta-analysis of literature and the development cohort; xₖ is the number of alleles of the kth SNP carried by an individual patient (xₖ=0, 1 or 2); and uₖ is the average number of alleles of the kth SNP reported for individuals included in large general population studies. Passing criteria may restrict the number of missing SNP calls such that the imputation of missing calls by the high or low risk allele(s) does not change the relative risk by more than 10%.

In some aspects, SNP coefficients can be estimated for the polygenic risk score.

In some embodiments, SNP coefficients can be estimated and standard errors for a plurality of pertinent SNPs can be obtained based on a development cohort. These coefficients can be designated {b_devk | k=1, 2, . . . , N_{SNP}}, and standard errors by {σ_devk | k=1, 2, . . . , N_{SNP}}, where N_{SNP} is the number of SNPs used. These values can be estimated from a single multivariate logistic regression model with breast cancer status as the dependent variable, and the following independent variables: N_{SNP} numeric variables representing allele counts for each of N_{SNP} SNPs {xk | k=1, 2, . . . , N_{SNP}}, age, ancestry, personal cancer history, and family cancer history. Age, ancestry, personal and family cancer history variables may be coded as described above. SNP coefficients can further be estimated by selecting literature-based coefficients {b_litk | k=1, 2, . . . , N_{SNP}}, and standard errors {σ_litk | k=1, 2, . . . , N_{SNP}}. Linkage disequilibrium between SNPs can be accounted for by co-estimating the effects in multivariate regression models, with one model for each gene.

Lastly, polygenic risk score coefficients can be calculated according to {bk | k=1, 2, . . . , N_{SNP}} from a meta-analysis of development cohort and literature-based coefficients. Polygenic risk score coefficients may be calculated as weighted averages of development cohort and literature coefficients with weights inversely proportional to squared standard errors. The ratio of squared standard errors can be replaced with the median value.

More specifically, for a plurality of SNPs, and with non-missing σ_litk values, a median ratio can be calculated according to Equation IV.

$$median\_ratio = median of [ {( \frac{{σ\_lit}_{1}}{{σ\_dev}_{1}} )}^{2} , {( \frac{{σ\_lit}_{2}}{{σ\_dev}_{2}} )}^{2} , … , {( \frac{{σ\_lit}_{Nsnp}}{{σ\_dev}_{Nsnp}} )}^{2} ] . \qquad Equation IV$$

where, for each k in 1 through N_{SNP}, bₖ was defined according to Equation V

$${b}_{k} = \frac{median\_ratio \times {b\_dev}_{k} + {b\_lit}_{k}}{1 + median\_ratio} . \qquad Equation V$$

In further aspects, the informativeness of each SNP can be calculated.

The informativeness of a SNP may be a function if its effect size, and its general population allele frequency. For each k in 1 through N_{SNP}, informativeness of the kth SNP can be calculated according to Equation VI.

$$2 \times {b}_{k}^{2} \times \frac{1}{2} {u}_{k} \times ( 1 - \frac{1}{2 {u}_{k}} ) . \qquad Equation VI$$

In additional aspects, SNPs may be ordered by informativeness. By designation, b₁ may denote the most informative SNP, b₂ the second most informative SNP, and so on.

Chi-square likelihood ratio test (LRT) statistics can be calculated to evaluate the contribution of each SNP to the polygenic risk score (PRS). For SNPs from linked sets, only the single most informative representative SNP from each gene may be included, leaving N_{SNP} less one for evaluation. For each k in 1 through N_{SNP}, analyses can be made in a development cohort according to the following steps. First, calculate k-SNP PRS scores for all patients according to Equation VII.

PRSₖ=b₁(x₁−u₁)+b₂(x₂−u₂)+ . . . . +bₖ(xₖ−uₖ) Equation VII.

Secondly, construct a multivariate logistic regression model with breast cancer status as the dependent variable, and independent variables for PRSₖ, age, ancestry, personal cancer history, and family cancer history. Third, record the LRT statistic comparing the full model to the nested model with PRSₖ omitted.

In further aspects, SNPs for a PRS may be selected according to highest likelihood ratio test (LRT) value. All linked SNPs from a gene may be included if the representative SNP was selected for inclusion.

The identity of a plurality of SNPs incorporated into an 86-SNP score embodiment are shown in Table 1. Chromosomal positions are given according to hg19.

TABLE 1
SNPs incorporated into an 86-SNP polygenic risk score

| | | CH | | 86-SNP |
|---|---|---|---|---|
| VARIANT | SOURCE | R | POSITION | SCORE |
| rs616488 | Mavaddat 2015 | 1 | 10566215 | rs616488 |
| rs11552449 | Mavaddat 2015 | 1 | 114448389 | rs11552449 |
| rs11249433 | Mavaddat 2015 | 1 | 121280613 | rs11249433 |
| rs12405132 | Michailidou 2015 | 1 | 145644984 | rs12405132 |
| rs72755295 | Michailidou 2015 | 1 | 242034263 | rs72755295 |
| rs112710696 | Mavaddat 2015 | 2 | 19320803 | rs12710696 |
| rs4849887 | Mavaddat 2015 | 2 | 121245122 | rs4849887 |
| rs2016394 | Mavaddat 2015 | 2 | 172972971 | rs2016394 |
| rs1550623 | Mavaddat 2015 | 2 | 174212894 | rs1550623 |
| rs13387042 | Mavaddat 2015 | 2 | 217905832 | rs13387042 |
| rs16857609 | Mavaddat 2015 | 2 | 218296508 | rs16857609 |
| rs6762644 | Mavaddat 2015 | 3 | 4742276 | rs6762644 |
| rs4973768 | Mavaddat 2015 | 3 | 27416013 | rs4973768 |
| rs12493607 | Mavaddat 2015 | 3 | 30682939 | rs12493607 |
| rs6796502 | Michailidou 2015 | 3 | 46866866 | rs6796502 |
| rs1053338 | Michailidou 2015 | 3 | 63967900 | rs1053338 |
| rs9790517 | Mavaddat 2015 | 4 | 106084778 | rs9790517 |
| rs6828523 | Mavaddat 2015 | 4 | 175846426 | rs6828523 |
| rs10069690 | Mavaddat 2015 | 5 | 1279790 | rs10069690 |
| rs7726159 | Mavaddat 2015 | 5 | 1282319 | rs7726159 |
| rs2736108 | Mavaddat 2015 | 5 | 1297488 | rs2736108 |
| rs13162653 | Michailidou 2015 | 5 | 16187528 | rs13162653 |
| rs2012709 | Michailidou 2015 | 5 | 32567732 | rs2012709 |
| rs10941679 | Mavaddat 2015 | 5 | 44706498 | rs10941679 |
| rs889312 | Mavaddat 2015 | 5 | 56031884 | rs889312 |
| rs10472076 | Mavaddat 2015 | 5 | 58184061 | rs10472076 |
| rs1353747 | Mavaddat 2015 | 5 | 58337481 | rs1353747 |
| rs7707921 | Michailidou 2015 | 5 | 81538046 | rs7707921 |
| rs1432679 | Mavaddat 2015 | 5 | 158244083 | rs1432679 |
| rs11242675 | Mavaddat 2015 | 6 | 1318878 | rs11242675 |
| rs204247 | Mavaddat 2015 | 6 | 13722523 | rs204247 |
| rs17529111 | Mavaddat 2015 | 6 | 82128386 | rs17529111 |
| rs12662670 | Mavaddat 2015 | 6 | 151918856 | rs12662670 |
| rs2046210 | Mavaddat 2015 | 6 | 151948366 | rs2046210 |
| rs6964587 | Michailidou 2015 | 7 | 91630620 | rs6964587 |
| rs4593472 | Michailidou 2015 | 7 | 130667121 | rs4593472 |
| rs720475 | Mavaddat 2015 | 7 | 144074929 | rs720475 |
| rs9693444 | Mavaddat 2015 | 8 | 29509616 | rs9693444 |
| rs13365225 | Michailidou 2015 | 8 | 36858483 | rs13365225 |
| rs6472903 | Mavaddat 2015 | 8 | 76230301 | rs6472903 |
| rs2943559 | Mavaddat 2015 | 8 | 76417937 | rs2943559 |
| rs13267382 | Michailidou 2015 | 8 | 117209548 | rs13267382 |
| rs13281615 | Mavaddat 2015 | 8 | 128355618 | rs13281615 |
| rs11780156 | Mavaddat 2015 | 8 | 129194641 | rs11780156 |
| rs1011970 | Mavaddat 2015 | 9 | 22062134 | rs1011970 |
| rs10759243 | Mavaddat 2015 | 9 | 110306115 | rs10759243 |
| rs865686 | Mavaddat 2015 | 9 | 110888478 | rs865686 |
| rs7072776 | Mavaddat 2015 | 10 | 22032942 | rs7072776 |
| rs11814448 | Mavaddat 2015 | 10 | 22315843 | rs11814448 |
| rs10995190 | Mavaddat 2015 | 10 | 64278682 | rs10995190 |
| rs704010 | Mavaddat 2015 | 10 | 80841148 | rs704010 |
| rs7904519 | Mavaddat 2015 | 10 | 114773927 | rs7904519 |
| rs11199914 | Mavaddat 2015 | 10 | 123093901 | rs11199914 |
| rs2981579 | Mavaddat 2015 | 10 | 123337335 | rs2981579 |
| rs3817198 | Mavaddat 2015 | 11 | 1909006 | rs3817198 |
| rs3903072 | Mavaddat 2015 | 11 | 65583066 | rs3903072 |
| rs78540526 | Mavaddat 2015 | 11 | 69331418 | rs78540526 |
| rs554219 | Mavaddat 2015 | 11 | 69331642 | rs554219 |
| rs75915166 | Mavaddat 2015 | 11 | 69379161 | rs75915166 |
| rs11820646 | Mavaddat 2015 | 11 | 129461171 | rs11820646 |
| rs10771399 | Mavaddat 2015 | 12 | 28155080 | rs10771399 |
| rs17356907 | Mavaddat 2015 | 12 | 96027759 | rs17356907 |
| rs1292011 | Mavaddat 2015 | 12 | 115836522 | rs1292011 |
| rs11571833 | Mavaddat 2015 | 13 | 32972626 | rs11571833 |
| rs2236007 | Mavaddat 2015 | 14 | 37132769 | rs2236007 |
| rs2588809 | Mavaddat 2015 | 14 | 68660428 | rs2588809 |
| rs999737 | Mavaddat 2015 | 14 | 69034682 | rs999737 |
| rs941764 | Mavaddat 2015 | 14 | 91841069 | rs941764 |
| rs11627032 | Michailidou 2015 | 14 | 93104072 | rs11627032 |
| rs3803662 | Mavaddat 2015 | 16 | 52586341 | rs3803662 |
| rs17817449 | Mavaddat 2015 | 16 | 53813367 | rs17817449 |
| rs13329835 | Mavaddat 2015 | 16 | 80650805 | rs13329835 |
| chr17:29239529:D | Michailidou 2015 | 17 | 29230520 | rs62070643** |
| rs6504950 | Mavaddat 2015 | 17 | 53056471 | rs6504950 |
| rs745570 | Michailidou 2015 | 17 | 77781725 | rs745570 |
| rs527616 | Mavaddat 2015 | 18 | 24337424 | rs527616 |
| rs1436904 | Mavaddat 2015 | 18 | 24570667 | rs1436904 |
| rs6507583 | Michailidou 2015 | 18 | 42399590 | rs6507583 |
| rs8170 | Mavaddat 2015 | 19 | 17389704 | rs8170 |
| rs2363956 | Mavaddat 2015 | 19 | 17394124 | rs2363956 |
| rs4808801 | Mavaddat 2015 | 19 | 18571141 | rs4808801 |
| rs3760982 | Mavaddat 2015 | 19 | 44286513 | rs3760982 |
| rs2823093 | Mavaddat 2015 | 21 | 16520832 | rs2823093 |
| rs17879961 | Mavaddat 2015 | 22 | 29121087 | rs17879961 |
| rs132390 | Mavaddat 2015 | 22 | 29621477 | rs132390 |
| rs6001930 | Mavaddat 2015 | 22 | 40876234 | rs6001930 |

*Originally published variant substituted by LD SNP, only SNPs with R2 ≥ 0.9 are listed.
**SNP in LD with published variant.

### Cancer Methods and Treatment

Cancer therapy can include surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound including, for example, a biologic or exogenous active agent.

Examples of treatments include bariatric surgical intervention, physical therapy, diet, and diet supplementation.

Examples of a cancer biological therapy include adoptive cell transfer, angiogenesis inhibitors, bacillus Calmette-Guerin therapy, biochemotherapy, cancer vaccines, chimeric antigen receptor (CAR) T-cell therapy, cytokine therapy, gene therapy, immune checkpoint modulators, immunoconjugates, monoclonal antibodies, oncolytic virus therapy, and targeted drug therapy.

Examples of a cancer surgery include lumpectomy, partial mastectomy, total mastectomy, simple mastectomy, modified radical mastectomy, radical mastectomy, and Halsted radical mastectomy.

Examples of a cancer drug include drugs approved to prevent breast cancer including Evista (Raloxifene Hydrochloride), Raloxifene Hydrochloride, and Tamoxifen Citrate.

Examples of a cancer drug include drugs approved to treat breast cancer including, Abemaciclib, Abraxane (Paclitaxel Albumin-stabilized Nanoparticle Formulation), Ado-Trastuzumab Emtansine, Afinitor (Everolimus), Afinitor Disperz (Everolimus), Alpelisib, Anastrozole, Aredia (Pamidronate Disodium), Arimidex (Anastrozole), Aromasin (Exemestane), Atezolizumab, Capecitabine, Cyclophosphamide, Docetaxel, Doxorubicin Hydrochloride, Ellence (Epirubicin Hydrochloride), Enhertu (Fam-Trastuzumab Deruxtecan-nxki), Epirubicin Hydrochloride, Eribulin Mesylate, Everolimus, Exemestane, 5-FU (Fluorouracil Injection), Fam-Trastuzumab Deruxtecan-nxki, Fareston (Toremifene), Faslodex (Fulvestrant), Femara (Letrozole), Fluorouracil Injection, Fulvestrant, Gemcitabine Hydrochloride, Gemzar (Gemcitabine Hydrochloride), Goserelin Acetate, Halaven (Eribulin Mesylate), Herceptin Hylecta (Trastuzumab and Hyaluronidase-oysk), Herceptin (Trastuzumab), Ibrance (Palbociclib), Ixabepilone, Ixempra (Ixabepilone), Kadcyla (Ado-Trastuzumab Emtansine), Kisqali (Ribociclib), Lapatinib Ditosylate, Letrozole, Lynparza (Olaparib), Megestrol Acetate, Methotrexate, Neratinib Maleate, Nerlynx (Neratinib Maleate), Olaparib, Paclitaxel, Paclitaxel Albumin-stabilized Nanoparticle Formulation, Palbociclib, Pamidronate Disodium, Perjeta (Pertuzumab), Pertuzumab, Piqray (Alpelisib), Ribociclib, Talazoparib Tosylate, Talzenna (Talazoparib Tosylate), Tamoxifen Citrate, Taxotere (Docetaxel), Tecentriq (Atezolizumab), Thiotepa, Toremifene, Trastuzumab, Trastuzumab and Hyaluronidase-oysk, Trexall (Methotrexate), Tykerb (Lapatinib Ditosylate), Verzenio (Abemaciclib), Vinblastine Sulfate, Xeloda (Capecitabine), and Zoladex (Goserelin Acetate).

As used herein, the term “disease” includes any disorder, condition, sickness, ailment that manifests in, for example, a disordered or incorrectly functioning organ, part, structure, or system of the body.

As used herein, the term “sample” includes any biological sample that is isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between cells, including synovial fluid, gingival crevicular fluid, bone marrow, cerebrospinal fluid (CSF), saliva, mucous, sputum, semen, sweat, urine, or any other bodily fluids. A blood sample can include whole blood or any fraction thereof, including blood cells, red blood cells, white blood cells or leucocytes, platelets, serum and plasma.

As used herein, the term “subject” includes humans. Humans generally include women and men and others such as non-binary.

In some embodiments, this invention can provide methods for recommending therapeutic regimens, including withdrawal from therapeutic regiments.

In further embodiments, an odds ratio can provide a clinician with a prognostic picture of a subject's biological state. Such embodiments may provide subject-specific prognostic information, which can be informative for a therapy decision, and may also facilitate monitoring therapy response. Such embodiments may result in a surprisingly improved treatment, such as better control of a disease, or an increase in the proportion of subjects achieving amelioration of symptoms.

As used herein, the terms “biologic,” “biotherapy,” and/or “biopharmaceutical” can include pharmaceutical therapy products manufactured or extracted from a biological substance. A biologic can include vaccines, blood or blood components, allergenics, somatic cells, gene therapies, tissues, recombinant proteins, and living cells; and can be composed of sugars, proteins, nucleic acids, living cells or tissues, or combinations thereof.

As used herein, the terms “therapeutic regimen,” “therapy” and/or “treatment” can include any clinical management of a subject, as well as interventions, whether biological, chemical, physical, or a combination thereof, intended to sustain, ameliorate, improve, or otherwise alter the condition of a subject.

As used herein, the term “administering” can include the placement of a composition into a subject by a method or route that results in at least partial localization of the composition at a desired site such that a desired effect is produced. Routes of administration include both local and systemic administration. Generally, local administration results in more of the composition being delivered to a specific location as compared to the entire body of the subject, whereas, systemic administration results in delivery to essentially the entire body of the subject. “Administering” also includes performing physical actions on a subject's body, including physical therapy, as well as chiropractice, massage and acupuncture.

### Devices and Systems

As used herein, the term machine-readable storage medium can comprise, for example, a data storage material that is encoded with machine-readable data or data arrays. The data and machine-readable storage medium may be capable of being used for a variety of purposes, when using a machine programmed with instructions for using said data. Such purposes include storing, accessing and manipulating information relating to the risk of a subject or population over time, or risk in response to treatment, or for drug discovery for inflammatory disease. Data comprising genomic measurements can be implemented in computer programs that are executing on programmable computers, which may comprise a processor, a data storage system, one or more input devices, one or more output devices. Program code can be applied to the input data to perform the functions described herein, and to generate output information. Output information can then be applied to one or more output devices. A computer can be, for example, a personal computer, a microcomputer, or a workstation.

As used herein, the term computer program can be instruction code implemented in a high-level procedural or object-oriented programming language, to communicate with a computer system. The program may be implemented in machine or assembly language. The programming language can also be a compiled or interpreted language. Each computer program can be stored on storage media or a device such as ROM, or magnetic diskette, and can be readable by a programmable computer for configuring and operating the computer when the storage media or device is read by the computer to perform the described procedures. A health-related or genomic data management system can be considered to be implemented as a computer-readable storage medium, configured with a computer program, where the storage medium causes a computer to operate in a specific manner to perform various functions.

### CONCLUSION

All publications, patents and literature specifically mentioned herein are hereby incorporated by reference in their entirety for all purposes.

Words specifically defined herein have the meaning provided in the context of the present disclosure as a whole, and as are typically understood by those skilled in the art. As used herein, the singular forms “a,” “an,” and “the” include the plural.

While the present disclosure is described in conjunction with various embodiments, it is not intended that the present disclosure be limited to such embodiments. On the contrary, the present disclosure encompasses various alternatives, modifications, and equivalents, as will be appreciated by those of skill in the art.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In addition, the materials, methods, and examples herein are illustrative only and not intended to be limiting.

Although the foregoing disclosure has been described in some detail by way of illustration and examples for purposes of clarity of understanding, it will be understood by persons of skill in the art that various changes and modifications may be practiced within the scope of the invention and the appended claims.

### EXAMPLES

### Example 1: Comprehensive Breast Cancer Risk Prediction

An IRB-approved study included de-identified clinical records from 358,471 women of European ancestry who were tested clinically for hereditary cancer risk with a multi-gene panel.

A comprehensive risk prediction was based on analysis of CHEK2 PV carriers (N=4,331) and women negative for BC gene PV (N=353,681) who were tested between September 2013 and July 2019.

Risk estimates that incorporated CHEK2, a SNP-based score, and Tyrer-Cuzick elements were calculated using a method of fixed-stratification. Fixed-stratification accounted for correlations between risk factors in a manner equivalent to a multivariable co-estimation. Risk stratification was assessed in an independent cohort of CHEK2 carriers (N=459) who were tested after July 2019.

In this example, significant correlations of CHEK2 status with family history (p=4.1×10⁻¹⁷) and of the SNP-based score with family history among CHEK2 carriers (p=1.7×10⁻⁵) were detected.

For these factors, joint effects were co-estimated using the fixed-stratification method. In an independent cohort, 24.0% of CHEK2 carriers were categorized as low risk (<20%), and 62.6% were categorized as moderate risk (20-50%). For 13.4% of CHEK2 carriers, risk estimation incorporating the SNP-based score and the Tyrer-Cuzick elements generated breast cancer risks of greater than 50%, consistent with analysis with genes recognized as highly penetrant. The distribution of risk was approximately a bell shaped curve having median 36.3%, mean 36.0%, range of 0.7% to 75.6%, where Q1 was 21.0% and Q3 was 49.1%.

These results showed that the comprehensive risk assessment method of this invention can provide surprisingly accurate risk estimation as compared to conventional methods based on highly penetrant genes. In sum, these results showed that a pathogenic variant in CHEK2 rendered it to be a marker with usefulness equal to a highly penetrant non-deleteriously mutated breast cancer gene in its cohort.

These results further showed that in CHEK2 PV carriers, comprehensive risk assessment can inform individualized decision-making and lead to improved targeting of screening and prevention strategies. In sum, these results showed that a pathogenic variant in CHEK2 rendered it to be a marker with usefulness equal to a highly penetrant non-deleteriously mutated breast cancer gene in its cohort.

### Example 2: Comprehensive Breast Cancer Risk Prediction

Study criteria included 706 women of White/Non-Hispanic and/or Ashkenazi Jewish ancestry who were referred for hereditary cancer testing with a multigene panel at Myriad Genetic Laboratories between April 2017 and January 2020, and who had a deleterious mutation (DM) in the CHEK2 gene and tested negative for mutations in all of 10 other breast cancer (BC) associated genes on the panel (BRCA1, BRCA2, TP53, PTEN, STK11, CDH1, PALB2, ATM, NBN, BARD1).

Women were eligible for inclusion only if they were submitted from states that allow the research use of clinical samples after completion of genetic testing.

Women were only included if they had complete data for the 86 SNPs included in Myriad's PRS86 calculation, see Hughes E, Tshiaba P, Gallagher S, et al., Development and Validation of a Polygenic Risk Score to Predict Breast Cancer Risk, JCO Precision Oncology, 2020, accepted. Women were excluded from the analysis if they had a personal history of LCIS, atypical hyperplasia, or breast biopsy as these would influence TC calculations.

CHEK2 DM status was determined based on Myriad's CHEK2 DM classifications as of January 2020, and may differ from classifications used when women actually received hereditary cancer testing. Individuals were excluded from this analysis if they had a biallelic CHEK2 DM due to the increased risk associated with biallelic carriers compared to monoallelic carriers. Individuals were also excluded if they had multiple CHEK2 DMs due to an inability to determine phase in this analysis.

Women were included in this analysis either as BC-affected cases referred for hereditary breast and ovarian cancer (HBOC) testing (n=556), or as unaffected controls referred for hereditary colon cancer testing (n=150). HBOC cases were more likely to have a first-degree relative with breast cancer (33%) compared to the controls (17%).

Cohort breakdown and clinical characteristics are shown in Tables 2 and 3, respectively.

TABLE 2
Cohort Breakdown

| Breast Cancer | n | Percent of total |
|---|---|---|
| No-Controls | 150 | 21% |
| Yes-Cases | 556 | 79% |

TABLE 3
Clinical Characteristics

| | Controls | | Cases | |
|---|---|---|---|---|
| Clinical Characteristics | N = 150 | | N = 556 | |
| Age at testing (median, range) | 46 | (20-81) | 49 | (25-83) |
| Age at testing < 50 (n, %) | 99 | (66%) | 320 | (58%) |
| Ancestry (n, %) | — | | — | |
| Ashkenazi Jewish | 0 | (0%) | 1 | (<1%) |
| Ashkenazi Jewish & White Non-Hispanic | 2 | (1%) | 8 | (1%) |
| White Non-Hispanic | 148 | (99%) | 547 | (98%) |
| First Degree Relative with BC | — | | — | |
| Yes | 25 | (17%) | 182 | (33%) |
| No | 125 | (83%) | 374 | (67%) |

Regression analyses were conducted using R version 3.5.3. Odds ratios and confidence intervals are reported per unit standard deviation in women without BC. P-values were calculated from likelihood ratio chi-squared test statistics and are reported as two-sided.

Two separate logistic regressions for breast cancer prediction were performed. A first regression was performed using an 86 SNP score (Myriad PRS86), along with age at testing and Ashkenazi ancestry. A second regression was performed using the log-odds of a comprehensive risk score Comprehensive-RRS, along with age at testing (continuous) and Ashkenazi ancestry. Log odds were used to account for the nature of the risk distribution curve. The results for the two separate logistic regressions for breast cancer prediction are shown in Table 4.

TABLE 4
Two separate logistic regressions for breast cancer prediction

| Method | Odds ratio | 95% CI | p-value |
|---|---|---|---|
| PRS86 | 1.72 | 1.36-2.21 | 6.0 × 10⁻⁶ |
| Comprehensive-RRS | 2.05 | 1.54-2.76 | 5.9 × 10⁻⁷ |

As shown in Table 4, the comprehensive risk score Comprehensive-RRS had a p-value 10-fold lower than for the 86-SNP based result. Thus, the comprehensive risk score Comprehensive-RRS provided surprisingly increased accuracy for breast cancer risk estimation.

In a further comparison, a single multivariate logistic regression was performed for predicting breast cancer status which incorporated both PRS86 and Comprehensive-RRS (log-odds). The single multivariate logistic regression took into account age at testing and Ashkenazi ancestry as covariates. Effect sizes were again calculated as odds ratios per one-unit standard deviation. The results for the single multivariate logistic regression for breast cancer prediction are shown in Table 5.

TABLE 5
Single logistic regression for breast cancer prediction

| Method | Odds ratio | 95% CI | p-value |
|---|---|---|---|
| PRS86 | 1.27 | 0.91-1.79 | 0.16 |
| Comprehensive-RRS | 1.69 | 1.13-2.52 | 0.011 |

As shown in Table 5, the comprehensive risk score Comprehensive-RRS had a p-value more than 14-fold lower than for the 86-SNP based result. Thus, the comprehensive risk score Comprehensive-RRS provided surprisingly increased accuracy and greater discrimination for breast cancer diagnosis and risk estimation.

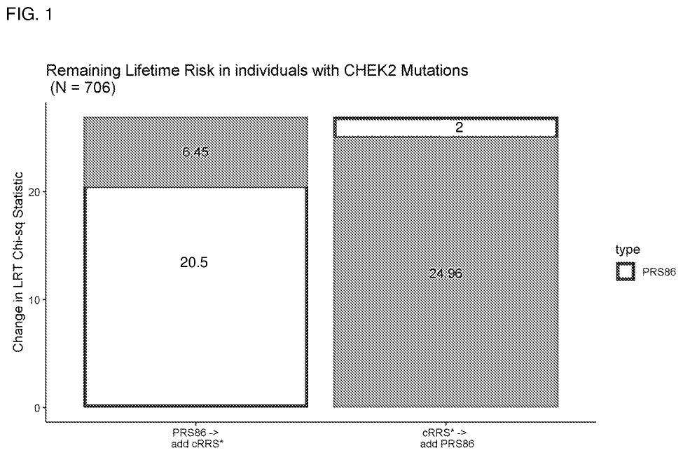

Referring to FIG. 1, an example is shown of the change in likelihood ratio test values (LRT) for breast cancer prediction. To compare the 86-SNP marker method and the Comprehensive-RRS method, one multivariate logistic regression was performed for predicting breast cancer status, with age at testing and Ashkenazi ancestry as covariates. Effect sizes were calculated as odds ratios per one-unit standard deviation. The remaining lifetime breast cancer risk was determined for study individuals having CHEK2 mutations. Referring to FIG. 1, the change in LRT on the left (20.5, 6.45) shows that breast cancer risk scores based on the 86-SNP marker method were significantly modified by including the Comprehensive-RRS method. However, the change in LRT on the right (24.96, 2) shows that breast cancer risk scores based on the Comprehensive-RRS method (cRRS*) were not significantly modified by adding the 86-SNP marker method. Thus, the comprehensive risk score Comprehensive-RRS provided surprisingly increased accuracy and greater discrimination for breast cancer diagnosis and risk estimation.

For SNP genotyping, genotyping calls from hybridization-based probes were validated using either Sanger sequencing or the IonTorrent Ampliseq platform as a comparator assay in 189 independent DNA samples with 100% concordance. Patients were called as having zero, one or two copies of each SNP based on observed read frequencies. SNPs with frequencies ranging from 0%-9% were called as zero copies; 20%-79% frequencies were called as one copy; and 90%-100% frequencies were called as two copies. An individual SNP was failed if its read frequency fell outside of the pre-specified thresholds, if it had less than 50×depth of coverage, or if a variant other than the expected wildtype or risk allele was observed.

### Example 3: Use of a Comparative 86-SNP Polygenomic Risk Estimation without Comprehensive Markers and Elements

An 86-SNP polygenic risk score was evaluated separately for carriers of pathogenic variants in BRCA1, BRCA2, CHEK2, ATM, PALB2, and non-carriers. Drawbacks of these data were that risk modification in CHEK2 carriers was the same with that observed in noncarriers. Also, the standardized odds ratios for carriers of BRCA1 and BRCA2 were less than that of CHEK2, ATM and PALB2 carrier populations. These unexpected results were likely due to the different effect sizes for the different genes, where confidence intervals for odds ratios overlapped, and where different score percentiles had widely different odds ratios, all of which reflected relative uncertainty and reduced accuracy. Thus, the use of an 86-SNP polygenomic risk estimation without comprehensive markers and elements was a comparative method.

An IRB-approved study included 152,012 women of European ancestry who were tested clinically for hereditary cancer risk with a multi-gene panel. An 86-SNP polygenic risk score was evaluated separately for carriers of pathogenic variants in BRCA1 (N=2,249), BRCA2 (N=2,638), CHEK2 (N=2,564), ATM (N=1,445) and PALB2 (N=906), and for non-carriers (N=141,160). Multivariable logistic regression was used to examine the association of the 86-SNP scores with invasive breast cancer after accounting for age and family cancer history. Effect sizes, expressed as standardized odds ratios (OR) with 95% confidence intervals (CIs), were assessed for carriers of each gene and for non-carriers. The 86-SNP score was strongly associated with breast cancer risk in BRCA1, BRCA2, CHEK2, ATM and PALB2 carrier populations (p<10⁻⁴). However, different effect sizes for different genes made further interpretation difficult.

The polygenic risk score was defined as a linear combination of centered risk alleles:

Polygenic Risk Score=b₁(x₁−u₁)+b₂(x₂−u₂)+ . . . . +b_{N}(x_{N}−u_{N})

where N was the total number of SNPs selected, the coefficient bₖ was the per-allele log OR for breast cancer association of the kth SNP estimated from meta-analysis of literature and the development cohort; xₖ was the number of alleles of the kth SNP carried by an individual patient (xₖ=0, 1 or 2); and uₖ was the average number of alleles of the kth SNP reported for individuals included in large general population studies. Passing criteria restricted the number of missing SNP calls such that the imputation of missing calls by the high or low risk allele(s) did not change the relative risk by more than 10%.

Associations with invasive breast cancer were evaluated in terms of p-values and ORs with 95% confidence intervals (CI) from multivariate logistic regression models constructed using R version 3.4.4 or higher (R Foundation for Statistical Computing, Vienna, Austria). ORs were reported per unit standard deviation of the polygenic risk score (PRS) in unaffected controls. P-values were calculated from likelihood ratio chi-square test statistics and reported as two-sided. Using multivariable logistic regression addresses the implicit bias in a genetic testing cohort where patients are selected for a qualifying factor, BC diagnosis or family history. Adjustment for factors related to ascertainment in a clinical testing population may enable the derivation of unbiased risk estimates.

All models included independent variables for age of first invasive breast cancer (BC) diagnosis or age at genetic testing if unaffected, personal history of non-BC, family history of any cancer and ancestry, European and/or Ashkenazi Jewish. Cases were women diagnosed with invasive breast cancer, with or without ductal carcinoma in situ (DCIS). Controls were BC cancer free at time of testing. Women diagnosed with DCIS were excluded from controls. In testing for a relationship between PRS and age, the multivariate model included an interaction term for PRS and age. An interaction test was also performed for PRS and carrier status, testing for a difference in PRS performance by gene. In this model a categorical variable represented the carrier status, non-carrier, BRCA1 pathogenic variant, BRCA2 pathogenic variant, etc., the PRS was standardized within each carrier group and an interaction term for PRS and carrier status was included.

Models included clinical variables for age, personal cancer history, family cancer history, and ancestry. Data were derived from the test request form submitted for hereditary genetic testing. Since clinical variables were also used to define eligibility for the study cohort, only women with complete clinical data are included in the study.

Age was coded in years as a continuous variable. The age of first diagnosis of invasive breast cancer was used for affected patients and age at the time of genetic testing for unaffected patients. Personal cancer variables were coded as binary, ever or never affected. Separate variables were coded for uterine/endometrial cancer, ovarian cancer, pancreatic cancer, stomach cancer, non-polyposis colorectal cancer, and adenomatous polyposis patients with ≥20 polyps.

All patients were tested for germline mutations for the following genes: APC, ATM, BARD1, BMPR1A, BRCA1, BRCA2, BRIP1, CDH1, CDK4, CDKN2A (p14ARF, p16), CHEK2, EPCAM, MLH1, MSH2, MSH6, MYH, NBN, PALB2, PMS2, PTEN, RAD51C, RAD51D, SMAD4, STK11, and TP53. Library preparation encompassed custom designed targeted next-generation sequencing (NGS) reagents for both exonic segments and additional DNA segments carrying informative breast cancer (BC) single nucleotide polymorphisms (SNPs). Long-range and nested PCR were applied to portions of the CHEK2 gene to exclude pseudogene sequences. Sequencing on HiSeq2500 or MiSeq instruments (Illumina Inc., San Diego, Calif.) identified both sequence variants and large rearrangements (deletions and duplications).

The primary analysis examined the association of the 86-SNP score with invasive BC in each gene carrier group. In exploratory analyses the performance of the 86-SNP score in carriers of CHEK2 1100delC or other CHEK2 PVs were compared. To test for the interaction with family history, either a binary variable (presence or absence of an affected first-degree relative) or the sum of relatives affected with invasive BC in a weighted relative count was used. To test for interaction with gene carrier status a categorical variable for non-carrier or gene-specific carrier status was created.

Familial cancers were coded as numeric counts of diagnoses, weighted according to degree of relatedness. A weight of 0.5 was used for each first-degree relative and 0.25 for each second-degree relative. Variables included ductal invasive breast cancer, lobular invasive breast cancer (LCIS), DCIS, male breast cancer, prostate cancer, and each of the personal cancer types listed above. Ancestries were coded as quantitative variables representing fractions of reported ancestries. For example, a patient who listed only Ashkenazi ancestry was coded with an Ashkenazi value of 1.0, and zero for European ancestries. A patient who reported European and Ashkenazi ancestries was coded with European and Ashkenazi values of 0.5.

To examine relative risks by percentiles of the 86-SNP score, the non-carrier and BRCA1, BRCA2, CHEK2, and ATM PV-positive cohorts were each binned into quintiles based on the 86-SNP score. The PALB2 cohort was binned into tertiles to account for the smaller sample size. The median percentile bin (33rd-66th percentile tertile for PALB2, 40-60th percentile quintile for all others) was set as the reference group in a model that also included the above described covariates.

Absolute lifetime risks of developing BC were calculated for unaffected study participants by combining the 86-SNP score-based risk with previously-published gene-specific risk estimates (for PV carriers) or lifetime BC risk estimates from Surveillance, Epidemiology, and End Results (SEER) 2009-2014 data (for non-carriers).

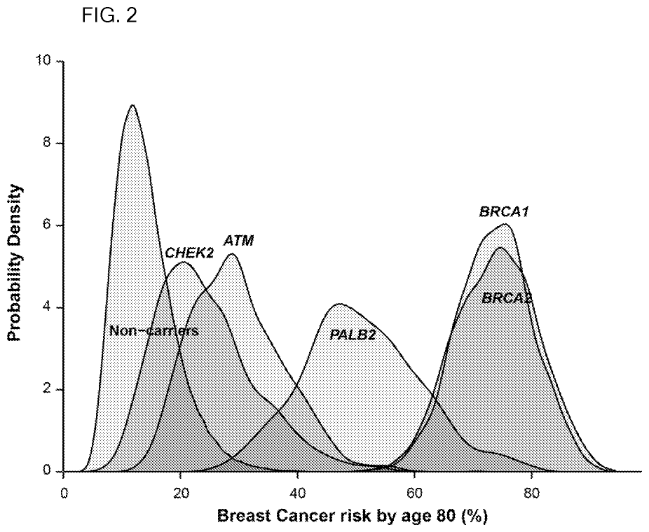

FIG. 2 shows lifetime breast cancer risk as probability density function against absolute risk estimates by age 80 for carriers of pathogenic variants (PV) in breast cancer associated genes as modified by an 86-SNP score method. For women with pathogenic variants (PV) in moderate-risk breast cancer genes CHEK2, ATM, and PALB2, point estimates were higher than for BRCA1/2 carriers. The interaction between the 86-SNP score and gene carrier type was significant. The most pronounced risk discrimination was observed for CHEK2 carriers, where the effect size was equivalent to the odds ratios observed in non-carriers and for the general population.

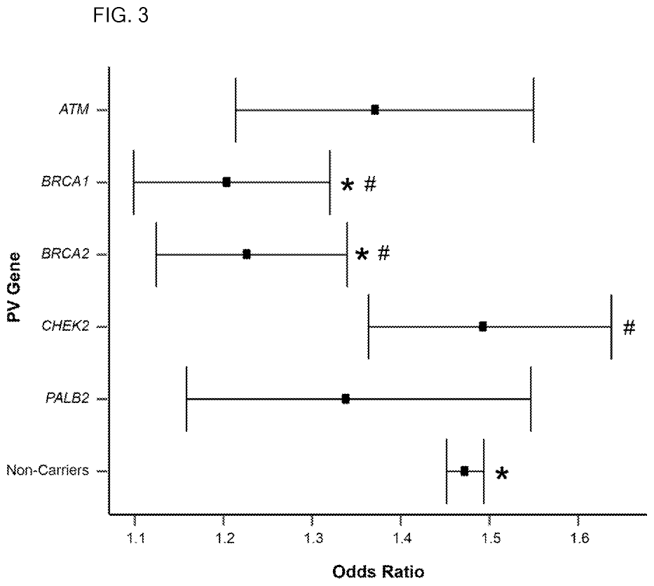

FIG. 3 shows standardized odds ratios for association between an 86-SNP score method and personal breast cancer history for carriers of each gene and non-carriers. FIG. 3 shows a forest plot of the standardized odds ratio for the association between the 86-SNP score and personal breast cancer history along with 95% confidence intervals for carriers of each gene and non-carriers.

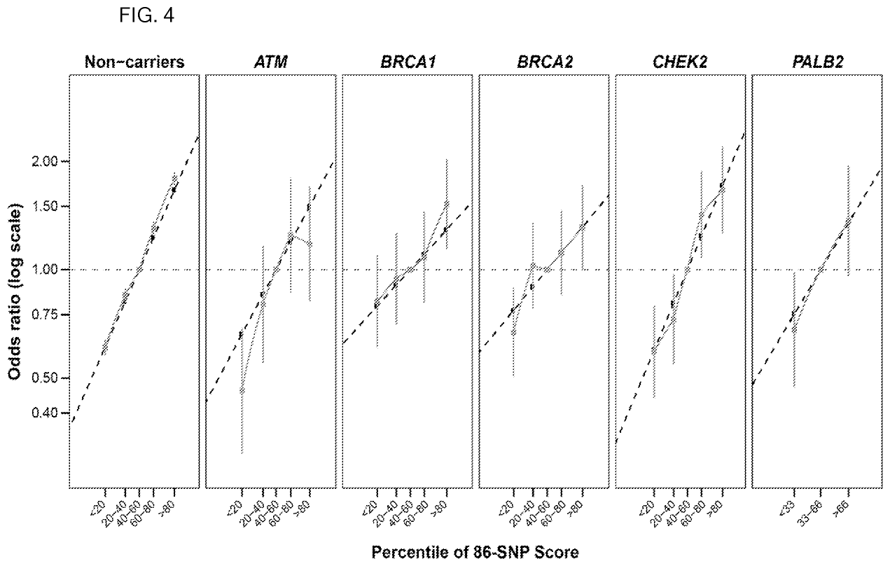

FIG. 4 shows observed (solid lines) versus expected (dashed lines) odds ratios per percentile of an 86-SNP score method by carrier gene.

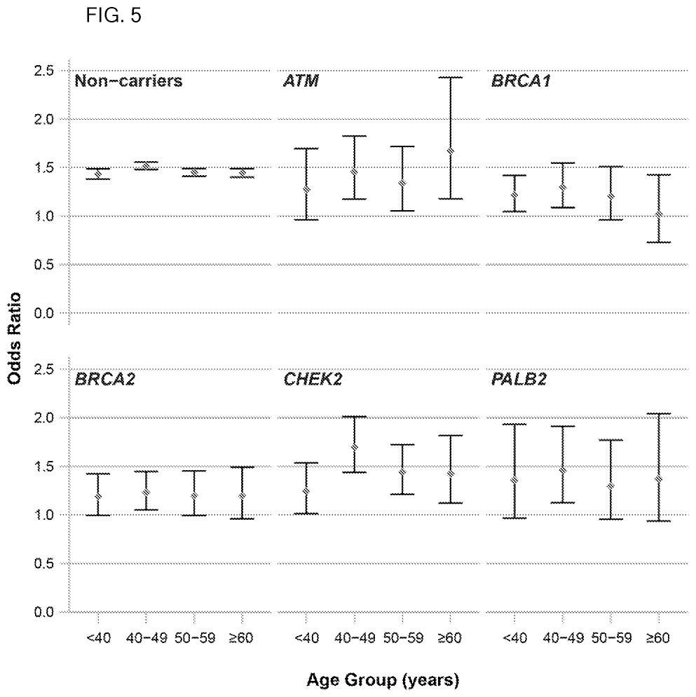

FIG. 5 shows odds ratios for the association of an 86-SNP score method with the risk of developing breast cancer by age bin and carrier gene.

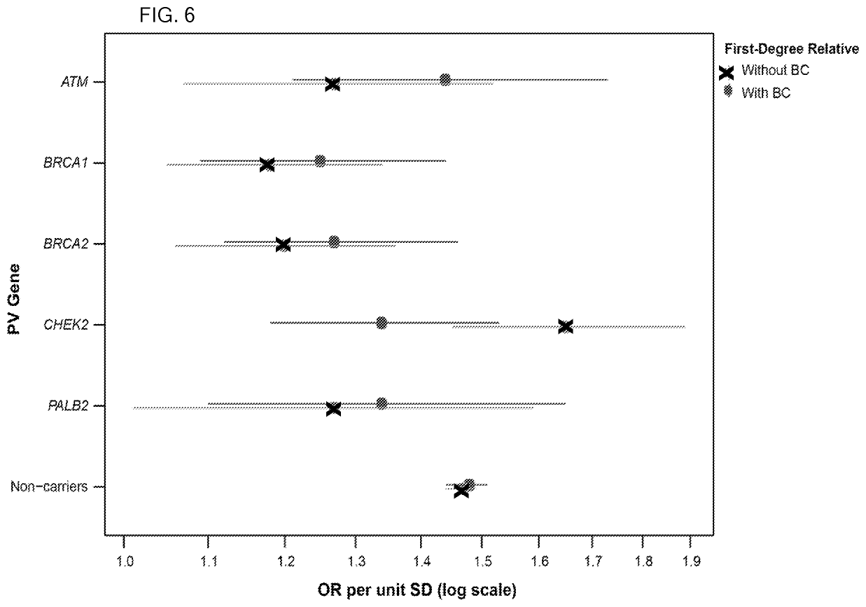

FIG. 6 shows odds ratios for the association of an 86-SNP score method with breast cancer risk by family history (X markers represent without breast cancer) and carrier gene (filled square markers represent with breast cancer).

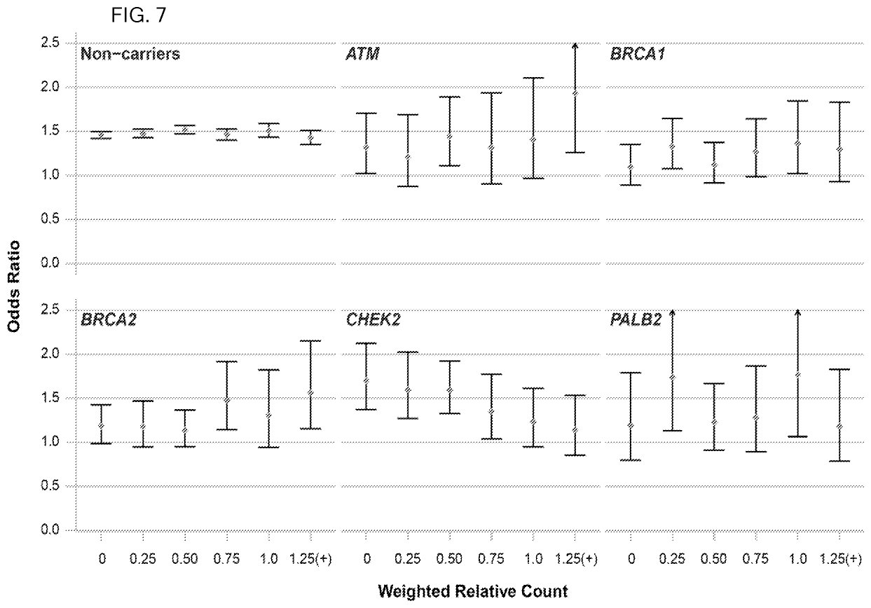

FIG. 7 shows odds ratios for the association of an 86-SNP score method with breast cancer risk by weighted relative count and carrier gene.

A summary of the clinical characteristics and demographic data of the study cohort is shown in Table 6.

TABLE 6
Summary of the clinical characteristics and
demographic data of the study cohort

| | Number of Women |
|---|---|
| Gene | with a PV^{a} |
| ATM | 1,445 |
| BARD1 | 331 |
| BRCA1 | 2,249 |
| BRCA2 | 2,638 |
| CDH1 | 92 |
| CHEK2 | 2,564 |
| NBN | 440 |
| PALB2 | 906 |
| PTEN | 49 |
| STK11 | 7 |
| TP53 | 131 |

^{a}Subjects with more than one PV were excluded from the 86-SNP score risk modification analysis.

ORs for developing breast cancer for the continuous 86-SNP score in carriers of CHEK2 1100delC and other CHEK2 PVs is shown in Table 7.

TABLE 7
ORs for developing breast cancer for the continuous 86-SNP score in
carriers of CHEK2 1100delC and other CHEK2 PVs

| CHEK2 PV type | N | OR (95 % CI) | p-value |
|---|---|---|---|
| 1100delC | 1426 | 1.38 (1.22-1.56) | 1.9 × 10⁻⁰⁷ |
| Other PV | 1138 | 1.67 (1.46-1.92) | 3.9 × 10⁻¹⁴ |

ORs for developing breast cancer for the continuous 86-SNP score by age bin and by carrier status for a PV in a BC-associated gene is shown in Table 8.

TABLE 8
ORs for developing breast cancer for the continuous 86-SNP score by
age bin and by carriers status for a PV in a BC-associated gene

| Pathogenic | Age | | | | |
|---|---|---|---|---|---|
| Variant | (years) | N | OR (95% CI | | p-value^{a} |
| None (non-carriers) | <40 | 44,350 | 1.43 | (1.38-1.49) | 1.2 × 10⁻⁸³ |
| | ≥40-<49 | 39,227 | 1.52 | (1.48-1.56) | 1.8 × 10⁻²³⁷ |
| | ≥50-<59 | 33,343 | 1.45 | (1.41-1.49) | 4.6 × 10⁻¹⁶⁶ |
| | ≥60 | 24,240 | 1.44 | (1.40-1.49) | 4.6 × 10⁻¹³⁰ |
| ATM | <40 | 448 | 1.27 | (0.96-1.70) | 0.09 |
| | ≥40-<49 | 431 | 1.46 | (1.17-1.82) | 5.9 × 10⁻⁴ |
| | ≥50-<59 | 329 | 1.34 | (1.05-1.72) | 0.02 |
| | ≥60 | 237 | 1.67 | (1.18-2.43) | 0.004 |
| BRAC1 | <40 | 1,086 | 1.22 | (1.05-1.42) | 0.01 |
| | ≥40-<49 | 567 | 1.30 | (1.09-1.55) | 0.004 |
| | ≥50-<59 | 371 | 1.20 | (0.96-1.51) | 0.11 |
| | ≥60 | 225 | 1.02 | (0.73-1.42) | 0.92 |
| BRCA2 | <40 | 992 | 1.19 | (0.99-1.42) | 0.06 |
| | ≥40-<49 | 693 | 1.23 | (1.05-1.45) | 0.009 |
| | ≥50-<59 | 529 | 1.20 | (0.99-1.45) | 0.06 |
| | ≥60 | 424 | 1.19 | (0.96-1.49) | 0.11 |
| CHEK2 | <40 | 817 | 1.25 | (1.01-1.54) | 0.04 |
| | ≥40-<49 | 753 | 1.70 | (1.44-2.01) | 1.2 × 10⁻⁹ |
| | ≥50-<59 | 607 | 1.44 | (1.21-1.72) | 2.6 × 10⁻⁵ |
| | ≥60 | 387 | 1.42 | (1.12-1.82) | 0.004 |
| PALB2 | <40 | 255 | 1.36 | (0.97-1.93) | 0.08 |
| | ≥40-<49 | 271 | 1.46 | (1.13-1.91) | 0.004 |
| | ≥50-<59 | 217 | 1.30 | (0.96-1.77) | 0.10 |
| | ≥60 | 163 | 1.37 | (0.94-2.04) | 0.10 |

^{a}p-value tests whether the OR is significantly different from 1.

ORs for developing breast cancer by BC affected status of a first-degree relative and by carrier status for a PV in a BC-associated gene is shown in Table 9.

TABLE 9
ORs for developing breast cancer by BC affected status of a first-degree
relative and by carrier status for a PV in a BC-associated gene

| | First-Degree Relative without Breast Cancer | | | First-Degree Relative with Breast Cancer | | |
|---|---|---|---|---|---|---|
| PV Gene | N | OR | 95% CI | N | OR | 95% CI |
| None (non- | 92,529 | 1.47 | (1.44-1.49) | 48,631 | 1.48 | (1.44-1.51) |
| carriers) | | | | | | |
| BRCA1 | 1,308 | 1.18 | (1.05-1.34) | 941 | 1.25 | (1.09-1.44) |
| BRCA2 | 1,493 | 1.20 | (1.06-1.36) | 1,145 | 1.27 | (1.12-1.46) |
| CHEK2 | 1,436 | 1.65 | (1.45-1.89) | 1,128 | 1.34 | (1.18-1.53) |
| ATM | 809 | 1.27 | (1.07-1.52) | 636 | 1.44 | (1.21-1.73) |
| PALB2 | 461 | 1.27 | (1.01-1.59) | 445 | 1.34 | (1.10-1.65) |

A summary of the clinical characteristics and demographic data of the study cohort is shown in Table 10.

TABLE 10
Summary of the clinical characteristics and demographic data of the study cohort

| | | | BRCA1 | | BRCA2 | | CHEK2 | | ATM | | PALB2 | |
|---|---|---|---|---|---|---|---|---|---|---|---|---|
| | Non- | | PV | | PV | | PV | | PV | | PV | |
| Variable | Carriers | | Carriers | | Carriers | | Carriers | | Carriers | | Carriers | |
| Total Patients | 141,160 | | 2,249 | | 2,638 | | 2,564 | | 1,445 | | 906 | |
| Age at Hereditary Cancer | 48 | (18, 84) | 4 | (18, 84) | 47 | (18, 84) | 48 | (18, 84) | 49 | (18, 84) | 51 | (18, 82) |
| Testing, Median (Range) | | | | | | | | | | | | |

BC History

| | | | | | | | | | | | | |
|---|---|---|---|---|---|---|---|---|---|---|---|---|
| Personal BC, N (%) | 28,928 | (20) | 828 | (37) | 897 | (34) | 914 | (36) | 486 | (34) | 401 | (44) |
| ≥1 First-or Second- | 100,216 | (71) | 1,700 | (76) | 2,003 | (76) | 1,972 | (77) | 1,101 | (76) | 720 | (79) |
| Degree Relative, N (%) | | | | | | | | | | | | |

Ancestry

| | | | | | | | | | | | | |
|---|---|---|---|---|---|---|---|---|---|---|---|---|
| Ashkenazi Jewish, N (%) | 2,924 | (2) | 69 | (3) | 59 | (2) | 24 | (1) | 16 | (1) | 8 | (1) |
| White/Non-Hispanic, N(%) | 134,819 | (96) | 2,115 | (94) | 2,504 | (95) | 2,504 | (98) | 1,404 | (97) | 886 | (98) |
| Ashkenazi Jewish and | 3,417 | (2) | 65 | (3) | 75 | (3) | 36 | (1) | 25 | (2) | 12 | (1) |
| White/Non-Hispanic, N (%) | | | | | | | | | | | | |

Modification of risk of development of breast cancer by an 86-SNP polygenic risk score in carriers of a pathogenic variant in five BC-associated genes is shown in Table 11.

TABLE 11
Risk of breast cancer for 86-SNP polygenic
risk score in carriers of a PV

| PV Cohort | N | OR | 95% CI | p-value |
|---|---|---|---|---|
| ATM | 1,445 | 1.37 | 1.21-1.55 | 2.6 × 10⁻⁷ |
| BRCA1 | 2,249 | 1.20 | 1.10-1.32 | 6.5 × 10⁻⁵ |
| BRCA2 | 2,638 | 1.23 | 1.12-1.34 | 4.2 × 10⁻⁶ |
| PALB2 | 906 | 1.34 | 1.16-1.55 | 6.2 × 10⁻⁵ |
| CHEK2 | 2,564 | 1.49 | 1.36-1.64 | 1.3 × 10⁻¹⁸ |
| Non-carriers | 141,160 | 1.47 | 1.45-1.49 | <5 × 10⁻³²⁴ |

Odds ratios for developing breast cancer by percentile of an 86-SNP PRS and by carrier status for a pathogenic variant in a BC associated gene is shown in Tables 12 and

TABLE 12
Risk of breast cancer for 86-SNP polygenic risk score in carriers of a PV

| 86-SNP | Non-Carriers | | ATM | | CHEK2 | |
|---|---|---|---|---|---|---|
| Score | OR | | OR | | OR | |
| Percentile | (95% CI) | p-value | (95% CI) | p-value | (95% CI) | p-value |
| ≤20 | 0.61 | 8.6 × 10⁻⁹⁰ | 0.46 | 1.7 × 10⁻⁴ | 0.59 | 5.6 × 10⁻⁴ |
| | (0.58-0.64) | | (0.31-0.69) | | (0.44-0.80) | |
| >20-≤40 | 0.85 | 3.4 × 10⁻¹² | 0.80 | 0.25 | 0.73 | 0.03 |
| | (0.81-0.89) | | (0.55-1.17) | | (0.54-0.97) | |
| >40-≤60^{a} | 1 | — | 1 | — | 1 | — |
| >60-≤80 | 1.30 | 6.4 × 10⁻³² | 1.25 | 0.23 | 1.42 | 0.01 |
| | (1.24-1.36) | | (0.87-1.80) | | (1.08-1.88) | |
| >80 | 1.79 | 1.5 × 10⁻¹⁶¹ | 1.18 | 0.38 | 1.67 | 3.0 × 10⁻⁴ |
| | (1.72-1.87) | | (0.82-1.71) | | (1.26-2.20) | |

| | |
|---|---|
| 86-SNP | PALB2 |

| Score | OR | OR |
|---|---|---|
| Tertile | (95% CI) | (95% CI) |
| ≤33 | 0.68 | 0.04 |
| | (0.47-0.98) | |
| >33-≤66^{a} | 1 | — |
| >66 | 1.37 | 0.09 |
| | (0.96-1.95) | |

^{a}The middle percentile was used as the referent; p-values are for the difference in effect size between the percentile of the 86-SNP score and the referent group.

Table 13: Risk of breast cancer for 86-SNP polygenic risk score in carriers of a PV

| 86-SNP | Non-Carriers | | BRCA1 | | BRCA2 | |
|---|---|---|---|---|---|---|
| Score | OR | | OR | | OR | |
| Percentile | (95% CI) | p-value | (95% CI) | p-value | (95% CI) | p-value |
| ≤20 | 0.61 | 8.6 × 10⁻⁹⁰ | 0.82 | 0.18 | 0.67 | 0.006 |
| | (0.58-0.64) | | (0.61-1.10) | | (0.50-0.89) | |
| >20-≤40 | 0.85 | 3.4 × 10⁻¹² | 0.94 | 0.70 | 1.02 | 0.86 |
| | (0.81-0.89) | | (0.70-1.26) | | (0.78-1.35) | |
| >40-≤60^{a} | 1 | — | 1 | — | 1 | — |
| >60-≤80 | 1.30 | 6.4 × 10⁻³² | 1.08 | 0.59 | 1.11 | 0.44 |
| | (1.24-1.36) | | (0.81-1.45) | | (0.85-1.46) | |
| >80 | 1.79 | 1.5 × 10⁻¹⁶¹ | 1.52 | 0.004 | 1.31 | 0.054 |
| | (1.72-1.87) | | (1.14-2.03) | | (1.00-1.72) | |

| 86-SNP | PALB2 | |
|---|---|---|
| Score | OR | OR |
| Tertile | (95% CI) | (95% CI) |
| ≤33 | 0.68 | 0.04 |
| | (0.47-0.98) | |
| >33-≤66^{a} | 1 | — |
| >66 | 1.37 | 0.09 |
| | (0.96-1.95) | |

^{a}The middle percentile was used as the referent; p-values are for the difference in effect size between the percentile of the 86-SNP score and the referent group.

Estimated lifetime breast cancer risk to age 80 and modification by an 86-SNP PRS is shown in Table 14.

TABLE 14
Estimated lifetime breast cancer risk to
age 80 and modification by an 86-SNP PRS

| | | Adjusted Lifetime Risk | | | | |
|---|---|---|---|---|---|---|
| | Gene-Based | Min | Q1 | Median | Q3 | Max |
| Gene | Risk (%) | (%) | (%) | (%) | (%) | (%) |
| ATM | 28.2 | 12.9 | 23.9 | 29.0 | 34.7 | 58.3 |
| BRCA1 | 73.5 | 53.1 | 69.4 | 73.8 | 77.9 | 91.5 |
| BRCA2 | 73.8 | 50.8 | 69.0 | 74.2 | 78.9 | 94.2 |
| CHEK2 | 22.1 | 6.6 | 18.1 | 23.0 | 29.1 | 70.6 |
| PALB2 | 50.1 | 26.2 | 44.4 | 50.3 | 57.3 | 79.2 |
| Non-carriers | 12.7 | 2.5 | 10.4 | 13.2 | 16.9 | 62.4 |

What is claimed is:

1. A method for assessing breast cancer risk in a subject having a pathogenic variant in a breast cancer associated gene, the method comprising:
measuring a genotype of the subject; and
calculating a polygenic risk score for breast cancer risk for the subject based on a plurality of breast cancer associated SNP markers of the genotype and additional variables for age, personal cancer history, family cancer history, and ancestry of the subject.

2. The method of claim 1, further comprising
calculating an adjusted TC risk (TC*) for the subject; and
assessing comprehensive breast cancer risk in the subject by combining the polygenic risk score and the adjusted TC risk.

3. The method of claim 2, further comprising validating the comprehensive breast cancer risk in a clinical cohort.

4. The method of claim 1, wherein the genotype is measured by NGS.

5. The method of claim 1, wherein the genotype is determined with a sequencing chip.

6. The method of claim 1, wherein the plurality of breast cancer associated SNP markers is from 10 to 10,000 SNP markers.

7. The method of claim 1, wherein the plurality of breast cancer associated SNP markers is from 50 to 200 SNP markers.

8. The method of claim 2, wherein the adjusted TC risk (TC*) is calculated to account for the presence of a CHEK2 DM according to Equation I:
TC*=1−(1−TC)^{exp(β}^{_{CHEK2}}^{+k}^{ᵢ}⁾, for family history strata i Equation I;
wherein
TC is the standard lifetime risk as calculated by Tyrer-Cuzick version 7.02;
β_{CHEK2} is a log-odds ratio for CHEK2 carriers as a predictor of breast cancer risk; and
kᵢ is a calibration constant for a specific family history strata i;
wherein subjects are divided into strata based on relative risk based on a comparison of individual risk due to familial cancer history compared to general population risk; and
wherein constants kᵢ can be calculated so that the mean of exp(β_{CHEK2}× [symbol] _{CHEK2}) within each strata is 1.

9. The method of claim 8, wherein the adjusted TC risk includes factors for age, body mass index, age at menarche, obstetric history, age at menopause, history of a benign breast condition that increases breast cancer risk such as hyperplasia, atypical hyperplasia, and/or LCIS, history of ovarian cancer, use of hormone replacement therapy, family history of breast and ovarian cancer, and Ashkenazi inheritance.

10. The method of claim 8, wherein the comprehensive breast cancer risk is a relative risk score (ComprehensiveRRS) for breast cancer risk made using an adjusted Tyrer-Cuzick risk and taking into account the presence of a CHEK2-DM according to Equation II;
ComprehensiveRRS=1−(1−TC*)^{exp(β}^{_{RRS}}^{+c}^{ᵢ}⁾ for family history strata i Equation II;
wherein
TC* is the adjusted Tyrer-Cuzick risk after accounting for the CHEK2 DM,
β_{RRS} is the log-odds per-unit log odds ratio of a polygenic SNP score from a multivariable logistic regression model with the effect of breast cancer family history fixed, and
cᵢ is a calibration constant for a specific family history strata i, calculated such that the average relative risk due to the polygenic SNP score was 1 within unaffected subjects from strata kᵢ.

11. The method of claim 1, wherein the genotype identifies a subject having the presence of a CHEK2-DM.

12. The method of claim 1, wherein the genotype identifies a subject who tested negative for mutations in breast cancer associated genes comprising BRCA1, BRCA2, TP53, PTEN, STK11, CDH1, PALB2, ATM, NBN, and BARD1.

13. The method of claim 1, wherein the calculating a polygenic risk score comprises a combination of centered risk alleles according to Equation III.
Polygenic Risk Score=b₁(x₁−u₁)+b₂(x₂−u₂)+ . . . . +b_{N}(x_{N}−u_{N}) Equation III;
wherein
N is the total number of SNPs selected;
coefficient bₖ is the per-allele log OR for breast cancer association of the kth SNP estimated from meta-analysis of literature and the development cohort;
xₖ is the number of alleles of the kth SNP carried by an individual patient which is 0, 1 or 2; and
uₖ is the average number of alleles of the kth SNP reported for individuals included in large general population studies.

14. The method of claim 13, wherein the total number of SNPs is 86.

15. The method of claim 3, wherein the clinical cohort includes women of white/non-hispanic and/or Ashkenazi Jewish ancestry.

16. A method for recommending a therapy for a subject having a pathogenic variant in a breast cancer associated gene and having breast cancer or at risk of breast cancer, the method comprising:
measuring a genotype of the subject;
calculating a polygenic risk score for breast cancer risk for the subject based on a plurality of breast cancer associated SNP markers of the genotype and additional variables for age, personal cancer history, family cancer history, and ancestry of the subject; and
recommending a therapy for the subject based on the risk of breast cancer indicating a need for treatment.

17. The method of claim 16, further comprising
calculating an adjusted TC risk for the subject; and
assessing comprehensive breast cancer risk in the subject by combining the polygenic risk score and the adjusted TC risk.

18. The method of claim 17, further comprising validating the breast cancer risk in a clinical cohort.

19. The method of claim 16, wherein the therapy is one of:
a therapy for the disease;
a monitoring period followed by a therapy for the disease;
a tapering of a therapy for the disease.

20. The method of claim 16, wherein the therapy is one or more of surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound.

21. A method for identifying a subject having breast cancer who benefits from a treatment, the method comprising:
measuring a genotype of the subject;
calculating a polygenic risk score for breast cancer risk for the subject based on a plurality of breast cancer associated SNP markers of the genotype and additional variables for age, personal cancer history, family cancer history, and ancestry of the subject; and
identifying the subject having the cancer who benefits from a treatment for the cancer based on the cancer risk indicating a need for treatment.

22. The method of claim 21, further comprising
calculating an adjusted TC risk for the subject; and
assessing comprehensive breast cancer risk in the subject by combining the polygenic risk score and the adjusted TC risk.

23. The method of claim 22, further comprising validating the breast cancer risk in a clinical cohort.

24. The method of claim 21, wherein the therapy is one of:
a therapy for the disease;
a monitoring period followed by a therapy for the disease;
a tapering of a therapy for the disease.

25. The method of claim 21, wherein the therapy is one or more of surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound.

26. A method for treating a disease in a subject in need thereof, the method comprising:
measuring a genotype of the subject;
calculating a polygenic risk score for breast cancer risk for the subject based on a plurality of breast cancer associated SNP markers of the genotype and additional variables for age, personal cancer history, family cancer history, and ancestry of the subject, wherein the risk score indicates a need for treatment; and
administering to the subject one of:
a therapy for the disease;
a monitoring period followed by a therapy for the disease;
a tapering of a therapy for the disease.

27. The method of claim 26, wherein the therapy is a cancer therapy selected from one or more of surgery, cryoablation, radiation therapy, bone marrow transplant, chemotherapy, immunotherapy, hormone therapy, stem cell therapy, drug therapy, biological therapy, and administration of a pharmaceutical, prophylactic or therapeutic compound.

28. A method for monitoring a response of a subject having breast cancer, the method comprising:
measuring a genotype of the subject;
calculating a polygenic risk score for breast cancer risk for the subject based on a plurality of breast cancer associated SNP markers of the genotype and additional variables for age, personal cancer history, family cancer history, and ancestry of the subject.

29. The method of claim 28, further comprising
calculating an adjusted TC risk for the subject; and
assessing comprehensive breast cancer risk in the subject by combining the polygenic risk score and the adjusted TC risk.

30. The method of claim 29, further comprising validating the breast cancer risk in a clinical cohort.

31. A method for prognosing a subject having breast cancer, the method comprising:
measuring a genotype of the subject;
calculating a polygenic risk score for breast cancer risk for the subject based on a plurality of breast cancer associated SNP markers of the genotype and additional variables for age, personal cancer history, family cancer history, and ancestry of the subject; and
prognosing the subject as having a poor prognosis for the disease based on the risk score.

32. The method of claim 31, further comprising
calculating an adjusted TC risk for the subject; and
assessing comprehensive breast cancer risk in the subject by combining the polygenic risk score and the adjusted TC risk.

33. The method of claim 32, further comprising validating the breast cancer risk in a clinical cohort.

34. A system for assessing risk of a disease in a subject, the system comprising:
a processor for receiving a genotype of the subject;
one or more processors for carrying out the steps:
calculating a polygenic risk score for breast cancer risk for the subject based on a plurality of breast cancer associated SNP markers of the genotype and additional variables for age, personal cancer history, family cancer history, and ancestry of the subject; and
calculating an adjusted TC risk for the subject;
assessing comprehensive breast cancer risk in the subject by combining the polygenic risk score and the adjusted TC risk; and
a display for displaying and/or reporting the risk score.

35. A non-transitory machine-readable storage medium having stored therein instructions for execution by a processor which cause the processor to perform the steps of a method for assessing risk of a disease in a subject, the method comprising:
receiving a genotype of the subject;
calculating a polygenic risk score for breast cancer risk for the subject based on a plurality of breast cancer associated SNP markers of the genotype and additional variables for age, personal cancer history, family cancer history, and ancestry of the subject;
calculating an adjusted TC risk for the subject;
assessing comprehensive breast cancer risk in the subject by combining the polygenic risk score and the adjusted TC risk; and
sending to a processor output for displaying and/or reporting the risk score.
